(19) United States
(12) Patent Application Publication
(10) Pub. No.: US 2016/0166807 A1
(43) Pub. Date: Jun. 16, 2016

(54) CANNULA-NEEDLE FOR INTRAVENOUS CATHETER, METHOD FOR THE MANUFACTURE THEREOF AND INFUSION DEVICE INCLUDING THE CANNULA-NEEDLE

(71) Applicant: LOREN MEDICAL DEVICE USA LTD, Willmingtion, New Castle County, DE (US)

(72) Inventors: Riccardo De Stefano, Lascari (IT); Silvestre Buscemi, Altavilla Milicia (IT); Giuseppe Buscemi, Palermo (IT)

(21) Appl. No.: 14/902,841

(22) PCT Filed: Jun. 27, 2014
(86) PCT No.: PCT/IB2014/062657

§ 371 Date: Jan. 4, 2016

(30) Foreign Application Priority Data
Jul. 4, 2013 (IT) CT2013A000016

(51) Int. Cl. A61M 25/06; B23K 26/04; B23K 26/382

(52) U.S. Cl. CPC A61M 25/0618; A61M 25/0606; B23K 26/382; B23K 26/048

(57) ABSTRACT

Certain exemplary embodiments provide systems, methods, and/or devices configured to utilize a cannula-needle having a tubular body adapted to house thereinside a needle of a catheter. The cannula-needle has a peripheral wall extending along a longitudinal direction with a predetermined length from an open proximal end to a distal end, the distal end provided with a hole.

### CROSS-REFERENCE TO RELATED PATENT APPLICATIONS

This application claims priority from PCT Application PCT/IB2014/062657, filed Jun. 27, 2014, titled “CANNULA-NEEDLE FOR INTRAVENOUS CATHETER, METHOD FOR THE MANUFACTURING THEREOF AND INFUSION DEVICE INCLUDING THE CANNULA-NEEDLE,” which is incorporated herein by reference in its entirety.

### BACKGROUND

1. Field

The present invention finds application in the field of medical devices for the introduction of fluids into the human body and particularly relates to a cannula-needle for intravenous catheter adapted to be used with a needle for intravenous introduction of medical fluids inside the human body.

The invention also relates to a method for manufacturing such a cannula-needle and an infusion device for medical fluids comprising the cannula-needle.

2. Brief Description

Cannulas for intravenous catheters, also defined cannula-needles, are medical devices designed to achieve in a fast and practical way a venous access in a patient's body that is both stable and usable for prolonged times for the injection of drugs or other medical liquids in a vein.

The main aim of the cannulas is to keep always available the venous access, avoiding having always to create a new one for each injection through the needle or intravenous catheter or having to leave the intravenous needle into the vein.

Typically, the cannula-needles comprise a tubular body, generally in a polymeric material such as Teflon®, designed to house thereinside the needle of the catheter in a coaxial position and having the distal end tapered and provided with a hole for delivering the fluids to be administered.

The intravenous needle will be dimensioned to protrude with its distal tip from the hole so as to penetrate into the venous access and to be removed at the end of injection, while the tubular body of the cannula-needle will remain in situ.

In this way, the cannula-needle, possibly fixed to the patient's skin via a patch, make the venous access always available and stable, without risk of compromising it by moving the patient, thus being usable for prolonged times and for a number of repeated fluids administrations.

The proximal end of the cannula-needle will instead be coupled to a connecting element for the temporary fixing of the cannula-needle to a syringe or an infusion line for the infusion of fluids.

The currently used cannula-needles essentially differ with each other for the size, in particular the length and the caliber.

In particular, the choice of the caliber, whose measure unit is the Gauge (GA), decreasing with the increasing of the caliber, affects the infusion speed of the medical fluid.

The most frequently used cannula-needles are those with caliber of GA 18, 32 mm long, 1.3 mm in circumference, 103 ml/min flow rate.

Cannula-needles with caliber of 20 GA, 32 mm long, 1.1 mm circumference, 67 ml/min flow rate are normally used for difficult veins, while for pediatric uses cannula-needles with a caliber of 22 GA, 25 mm long, 0.9 mm circumference, 42 ml/min flow rate are particularly used.

As evident, to a larger caliber, and then at a lower gauge, corresponds a greater infusion rate and therefore the cannula-needles with lower caliber are preferred in case it is necessary to inject large quantities of fluid in a relatively short time, as for example, in emergency situations with the patient in a state of shock.

However, the cannula-needles of the highest caliber result in more pain for the patient, both during the implementation of the venous access and during therapy, are more difficult to be inserted and have a higher risk of rupture of the vein.

A further drawback of the traditional cannula-needles is due to the fact that, since each of them is suitable to be used for a limited range of flows, it is required the availability of a high number of cannula-needles with different calibers and/or lengths, to cover all values of flow rate and speed of infusion.

US2006100583 discloses a cannula for intravenous catheter which has, in addition to the dispensing hole at the distal end, a plurality of holes formed on its peripheral wall in not aligned positions, in order to increase the maximum total flow rate of fluid that can be supplied and to obtain a higher infusion speed.

However, the peripheral holes have a diameter between 1.7 mm and 2.5 mm, which though lower than that of the distal hole, it is still comparable to the same and not suitable for cannulas having reduced caliber.

Moreover, the large diameter of the peripheral holes implies that they are in relatively small numbers, in particular less than 10, so that they are distributed along the cannula at a relatively large distance that causes the fluid to be supplied in a localized and non-homogeneous way along the extension of the cannula.

From WO2011/091275 an intravenous catheter is known which has a caliber of 18-20-22 GA and length between 2 and 6 cm with a distal hole and lateral passages arranged at a distance from the distal end between 1 and 12 mm.

In two particular embodiments, the diameter of the single circular passages is equal to 75 μm and 100 μm, with a total area greater than that of the distal hole, in order to slow down the speed of the flow in the catheter and allow the supplying of fluid especially from the walls.

As matter of fact, such a catheter is designed to be used for the infusion of a contrast fluid inside the human body, particularly in operations of angiography.

For this reason, its object is to maximize the delivery of the flow from the lateral walls to obtain a greater spread, and is not designed to have a regular and uniform distribution of the flow along the entire development of the catheter.

A further example of catheter adapted to be used in angiography is disclosed in WO01/51116, which catheter is defined by a flexible tube having a rounded distal end that does not allow its use as a needle and which has a very small distal hole to promote the supplying of the contrast fluid predominantly by the side passages, arranged along a limited portion of the whole longitudinal development of the catheter, always in order to maximize the lateral spread of the liquid.

Further examples of angioplasty catheters provided with a distal hole and lateral passages are disclosed in U.S. Pat. No. 4,801,297 and WO98/33544, which have one or more of the above drawbacks.

### SUMMARY

The object of the present invention is to overcome the above drawbacks, providing a cannula-needle for intravenous catheter that is particularly effective and simple to use.

A particular object is to provide a cannula-needle for intravenous catheters which allows to increase the maximum flow rate that can be supplied as compared to cannula-needle of equal size and that allows to supply the flow uniformly along its extension.

Still another object is to provide a cannula-needle for intravenous catheters that is efficient in a wide range of flow rates adapted to supplied to be used with a greater number of intravenous needles and on different types of patients.

Still another object is to provide a cannula-needle for intravenous catheters that has relatively reduced caliber to reduce the invasiveness in the body of the patient.

A particular object is to provide a cannula-needle for intravenous catheters which is structurally stable.

Not last object of the present invention is to make available a method for manufacturing such a cannula-needle that is particularly fast and accurate.

These objects, as well as others which will appear more clear hereinafter, are achieved by a cannula-needle for intravenous catheter which, according to claim 1, comprises a tubular body adapted to house thereinside the needle of the catheter and having a peripheral wall that extends along a longitudinal direction with a predetermined length from an open proximal end adapted to be anchored to the connection element of the catheter to a distal end, tapered and provided with a hole for the partial exit of the needle.

The peripheral wall includes at least one pair of elongated passages substantially longitudinal and diametrically opposite to each other, each having a minimum dimension of the order of tenths of millimeter and between ⅓ and a 1/7 of the maximum diameter of said distal hole and an overall longitudinal extension greater than 50% of said predetermined length of said peripheral wall to define a respective section for the passage of fluid having overall extension close to that of said distal hole and obtain a uniform distribution of the supplied fluid flow along the longitudinal extension of said tubular body with an increased total flow rate.

By this way, the passages will have a more uniform and regular extension but with an overall section however large to sensibly increase the maximum flow rate to be supplied, while maintaining reduced the dimensions of the cannula-needle and in particular the caliber.

Consequently, the cannula-needle may be less invasive and will also be capable of supply the fluid to be injected in a more uniform along its longitudinal extension.

Moreover, making passages with higher extension but with a minimum size extremely contained will avoid compromising the structural stability of the cannula-needle, which also make it easier and safe to handle for ease of insertion within the venous access.

The use of cannula-needle of smaller sizes will allow a healthcare operator to make use of intravenous needles of a smaller caliber and thus more easily find the vein, this being a condition particularly beneficial for pediatric patients or for patients under chemotherapy having severe venous problems.

In the present text, the terms proximal and distal indicate, in the usual manner, the parts of the cannula-needle respectively closest and farthest with respect of an operator during use of the cannula-needle on a patient.

According to a further aspect of the invention there is provided a method for manufacturing a cannula-needle for intravenous catheters which, according to claim 11, comprises a step of forming a plurality of passages on the peripheral wall of the tubular body of the cannula-needle for fluid supplying.

The method is characterized in that said step of forming the holes is carried out by generating and orienting a laser beam on said peripheral wall.

Preferably, said step of generating and orienting the laser beam may comprise a first step of adjusting the position of said tubular body with respect to the generator of said beam by adjusting the laser beam with a power less than its maximum power and with insufficient value to pierce said the tubular body and a second step of increasing the power of said laser beam to form the hole, possibly being a step being provided for adjusting the distance of said tubular body from the focal point of said beam for the variation of the diameter of said hole.

This operating method allows to have extremely precise passages and also with minimum size, of the order of microns or tenths of millimeter, without compromising the structural stability of the cannula.

Furthermore, the adjustment of the power of the laser beam will allow, in the case in which the holes have to be formed, to align themselves in a precise manner.

According to yet another aspect of the invention a device for infusion of medical fluids is provided according to claim 14.

Advantageous embodiments of the invention are achieved according with the appended claims.

### BRIEF DESCRIPTION OF THE DRAWINGS

Further features and advantages of the invention will become more apparent in light of a detailed description of some preferred but not limiting embodiments of a cannula-needle according to the invention, shown as a non-limiting example with the aid of the appended drawings wherein:

FIG. 1 is a perspective view of a catheter type infusion device for a fluid provided with a cannula-needle according to the invention;

FIG. 2 is an enlarged front view of a detail of the cannula-needle of FIG. 1;

FIG. 3 is an enlarged front view of a detail of a cannula-needle in a second preferred embodiment;

FIG. 4 is a front view of a cannula-needle in a third preferred embodiment;

FIG. 5 is a front view of a cannula-needle in a fourth preferred embodiment;

FIG. 6 is an enlarged front view of a detail of the cannula-needle of FIG. 5.

### DETAILED DESCRIPTION

FIG. 1 shows a device for the intravenous infusion of medical fluids, globally indicated with 1, provided with a cannula-needle 2 according to the present invention.

In the example of the figure, the device 1 is provided with a coupling element 3 adapted to be coupled with a syringe or other similar infusion device, not shown because of known type, adapted to contain and dispense a medical fluid in a controlled manner, such as a drug, a saline solution or the like.

In a known manner the fluid to be supplied may be contained in a drip or similar container, also not shown as known per se.

The connecting element 3 comprises a first portion 4, or main portion, provided with an intravenous needle 5 for intravenous injection and adapted to connect to the syringe or infusion device, and a second portion 6 or secondary portion, adapted to be connect in a removable manner to the main portion 4 and to be attached to the cannula-needle 2.

The secondary portion 6 suitably comprises a hollow cylindrical body 7 for the passage of the intravenous needle 5 and provided with a pair of side flaps 8 for its gripping and handling by an operator.

In turn, the cannula-needle 2 has a tubular body 9 adapted to house thereinside the intravenous needle 5 and having a peripheral wall 10 that extends along a longitudinal direction L with a predetermined length l from an open proximal end 11 adapted to be anchored to the cylindrical body 7 of the secondary portion 6 of the connecting element 3 of the catheter 1 at a distal end 12 provided with a main distal hole 13 preferably circular or slightly elliptical for the partial exit of the intravenous needle 5.

The cannula-needle 2 will be preferably made of a polymeric material, such as Teflon® or the like.

A needle guard 14 may also be provided which is designed to cover the assembly consisting of cannula-needle 2 and intravenous needle 5, when not in use, for safety and hygiene reasons.

It is understood that the shown device 1 is purely illustrative since the cannula-needle 2 according to the present invention may be used with any type of needles and associated devices, without particular theoretical limitations.

In its most general and peculiar embodiment to the present invention, the cannula-needle 2 has a tubular body 9 having a tubular peripheral wall 10 tapering toward the distal end 12, which has at least a pair of elongated passages 15, 15′ substantially longitudinal and diametrically opposite to each other.

Each of these passages 15, 15′ has a minimum dimension d of the order of tenths of a millimeter, in particular between a few microns and a few tens of microns, measured along a direction Y substantially tangential to the peripheral wall 10 of the cannula-needle and orthogonal to the extension longitudinal direction L of the cannula 2.

Moreover, the passages 15, 15′ will be sized so that their sections or surfaces have overall extension A_{T} comparable with that of the distal hole 13 so as to provide an additional flow rate Q_{iT} of the supplied flow that will be comparable with the flow rate Q_{f} supplied by the distal hole 13, that is greater or less than this for only a few percentage points, as will appear more evident by some clarifying embodiments of the present invention.

In general, regardless of the form of the passages 15, 15′, their minimum size d will be between ⅓ and 1/7, preferably ⅕, of the maximum diameter ID of the distal hole 13.

Furthermore, each passage 15, 15′ will have a longitudinal extension e at least equal to 50% of the length l of the tubular body 10 and preferably greater than 60%, for example about to ⅔.

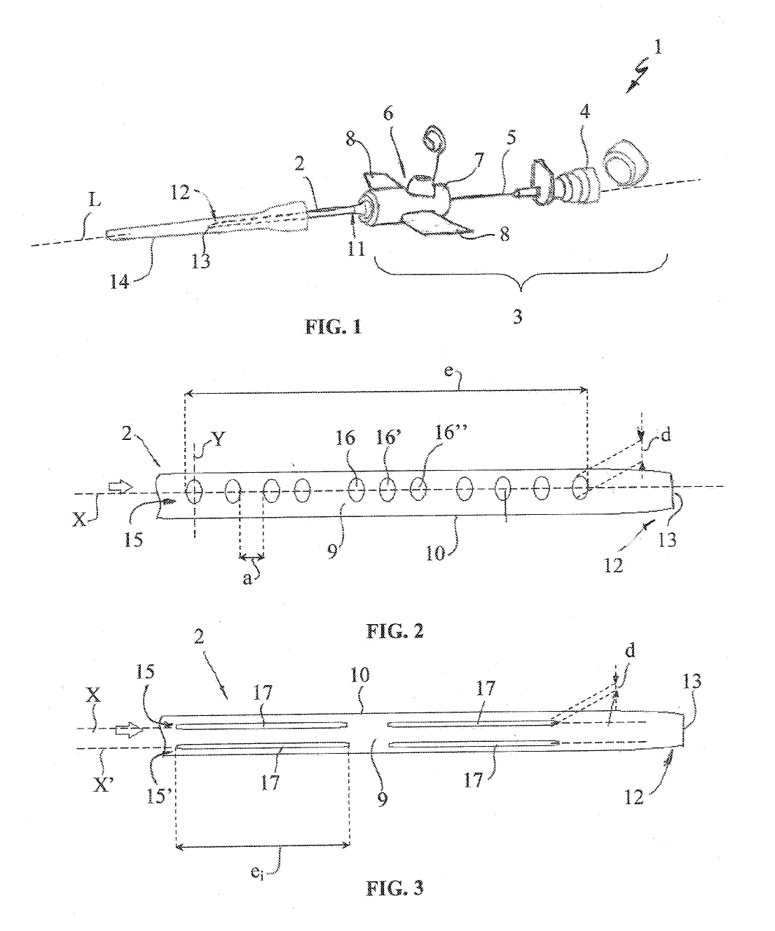

FIG. 2 shows a first preferred but not exclusive embodiments of the cannula-needle 2 according to the invention, wherein two passages 15, 15′ are provided which extend along respective longitudinal directions X, X′ parallel to the central axis L of the frustoconical tubular body 9, on diametrically opposite and symmetrical sides with respect to an axial plane it.

Each passage 15, 15′ is also defined by a plurality of peripheral holes 16, 16′, 16″, . . . similar with each other and substantially circular or slightly elliptical, which are longitudinally aligned and mutually offset from each other with predetermined and substantially constant axial distances a.

Hereinafter, for simplicity we will refer to the single peripheral hole 16, it being understood that everything will be referred to it will find in a substantially similar manner in the other peripheral holes 16′, 16″, . . . unless otherwise specified.

Each hole 16 will have a diameter φ defining the above minimum size d and the sum of their areas A will define the total surface extension A_{T} of the passages 15, 15′.

The number of peripheral holes 16 will be determined also as a function of the caliber GA of the cannula 2. However, it was experimentally observed that to obtain the total flow Qₜ between 50 ml/min and 100 ml/min, i.e. in the range of the more used values and that covers both the use in emergency situations, in which the maximum flow rates, and then the maximum infusion speed, are required, and the uses in the pediatric field, wherein the flow rates have lower values, will be sufficient to provide cannula-needles with GA22 and GA24.

The effectively flow rate supplied by the device 1 provided with the cannula-needle 2 may still be regulated upstream through the infusion tube of the drip tube or equivalent valve means, not shown.

For supplying these flow rates the distal hole 13 may have a diameter ID between 0.4 mm and 0.6 mm, so as to provide a partial flow rate Q_{f} between 25 ml/min and 45 ml/min.

In particular it was observed that to obtain the above flow rates it will be sufficient to provide cannulas with caliber G22 or G24, i.e. having a distal opening 13 with a diameter respectively of 0.6 mm and 0.4 mm, while the number of peripheral holes 16 for each passage 15, 15′ may be between 15 and 25.

It was also experimentally found that the ideal total number or minimum number nᵢ of peripheral holes 16 may be calculated using the following formula:

$${n}_{i} = int ( \frac{{Q}_{t} - {Q}_{f}}{{k}_{ρ} * A * {k}_{h}} )$$

where int denotes the integer part of the result included in parenthesis, Qₜ is the desired total nominal flow rate, Q_{f} is the nominal flow rate of the fluid supplied by the distal hole 13, A is the area of the single peripheral hole 16, considered at least theoretically as perfectly circular.

k_{ρ} is a parameter defined as a function of the used fluid and in particular of its density and may be set equal to 0.61 in the case of a fluid with a density equal or close to that of water, as in the case of physiological solutions.

kₕ is a parameter calculated as a function of the potential energy of the fluid and therefore it is dependent on the falling height of the supplied fluid, i.e. on the difference between the height of the starting point and the height of the supplying point.

For example, the parameter kₕ can be calculated with the following formula:

kₕ=\sqrt{2gΔh}

wherein g is the gravity acceleration and Δh is the abovementioned difference between the drop heights of the fluid, for example corresponding to the difference in height between the arm of the patient to which the cannula-needle 2 is applied and the bottle of the drip containing the fluid.

The actual number of holes nₑ may differ from that calculated number nᵢ and in particular will be increased to take account of any errors, so overestimating the number and the consequent maximum flow rate that may be supplied.

In particular, the actual number nₑ of peripheral holes 16 will be calculated with the following formula:

nₑ=int(nᵢ+ε)

wherein ε is a incremental coefficient adapted to oversize the cannula-needle and which may be directly proportional to the ideal number nᵢ.

The axial distance between the holes 16 in the same passage 15, 15′ may vary in function of the number of holes 16 and will generally be between 200 μm and 300 μm.

The length l of the tubular body 9 may be between 15 mm and 30 mm, preferably between 19 mm and 25 mm, with values directly proportional to the respective caliber GA.

Furthermore, the minimum distance of each passage 15, 15′ from the proximal end 11, which may be defined in the case of the figure by the distance between the proximal end 11 and the peripheral hole 16 nearest to the same, will preferably be between 5 mm and 7.5 mm, with values proportional to the length of the tubular body 9.

FIG. 3 shows a detail of a cannula 2 in a second preferred embodiment wherein each longitudinal passage 15, 15′ is defined by a series of substantially longitudinal slots 17, longitudinally aligned and mutually offset.

The width of the slots 17, as measured at right angles to the axial extension direction L, define the above minimum size d, while the length eᵢ of the single slots 17 will depend on their number and on the value of the partial and total flow rates to be supplied.

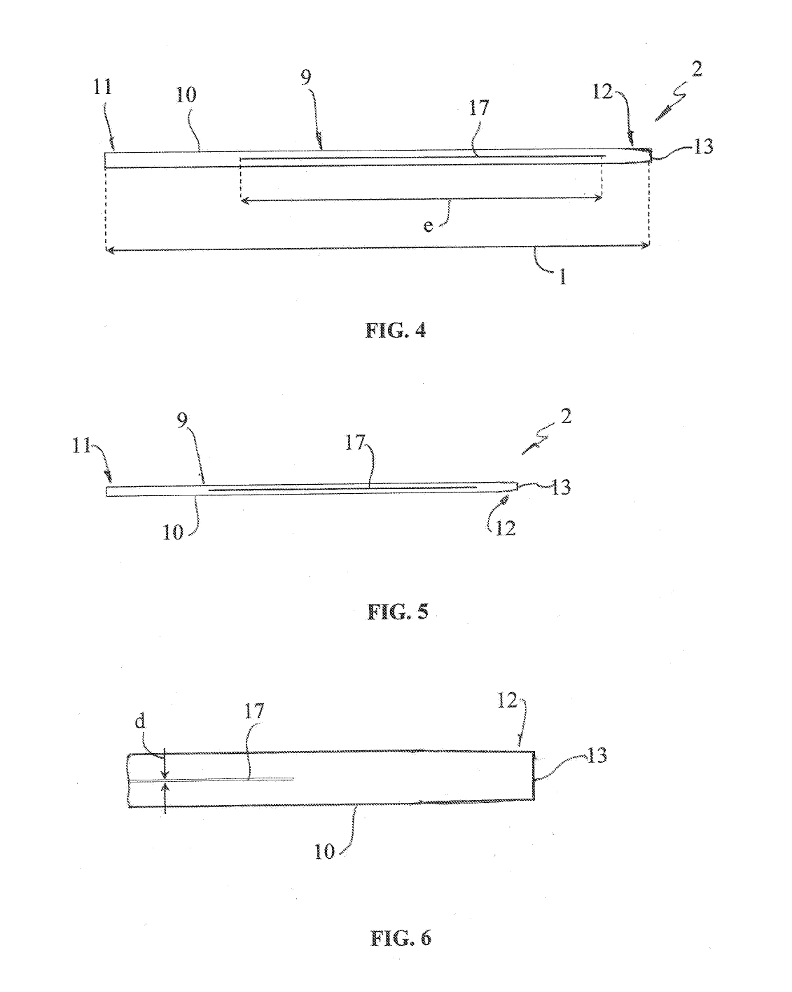

FIG. 4 shows a third embodiment of the cannula-needle 2 wherein each passage 15, 15′ is defined by a single continuous longitudinal slot 17.

The length of the tubular body 9 is substantially equal to 30 mm and each slot 17 has a longitudinal extension e equal to 20 mm with a distance from the proximal end equal to 7.5 mm and a distance from the distal end equal to 2.5 mm.

FIG. 4 and FIG. 5 show a fourth embodiment of the cannula-needle 2 wherein each passage 15, 15′ is defined by a single continuous longitudinal slot 17.

The length of the tubular body 9 is substantially equal to 20 mm and each slot 17 has a longitudinal extension e equal to 13 mm with a distance from the proximal end equal to 5 mm and a distance from the distal end equal to 2 mm.

Two examples are provided hereinafter for calculating the ideal number nᵢ of the peripheral holes 16 to be formed on the peripheral wall 10 of the tubular body 9, respectively for a cannula-needle 2 with caliber GA22, i.e. with a distal hole 13 having diameter ID equal to 0.6 mm and a maximum output flow rate Qₜ of 100 ml/min, and a cannula-needle 2 with caliber GA24, wherein the distal hole 13 has an inner diameter ID of 0.4 mm and a maximum output flow rate Qₜ of 50 ml/min.

In both cases it is assumed that the diameter d of the peripheral holes 16 is equal to ⅕ of the diameter ID of the distal hole 13, so as to avoid excessive weakening of the structure of the cannula-needle 2 and a possible breakage.

Furthermore, it is assumed that the fluid has density close to that of water, setting the parameter k_{ρ} equal to 0.61 and that Δh is equal to 1000 mm.

For the GA22 caliber the diameter d of the peripheral holes 16 will be equal to 0.12 mm, and then the radius r equal to 0.06 mm. Therefore the area A of each hole 16 will be equal to 0.0113 mm².

The flow rate Qi supplied by each single hole 16 will be given by the formula

Qᵢ=0.61*0.0113*√(2*9810*1000)=30.5322 mm³/s=1.83 cm³/min

The flow rate Q_{f} that may be supplied by the distal hole 13 will instead be equal to 42 ml/min and therefore the peripheral holes 16 along the cannula 2 may allow a flow rate Q_{iT} equal to

Q_{iT}=(100−42) ml/min=58 ml/min

The number nᵢ of peripheral holes 16 will therefore be

nᵢ=int(58/1.83)=31.

The actual number nₑ of holes 16 may be nₑ=40, with 20 peripheral holes 16 for each passage 15, 15′, so as to compensate possible errors and oversize the system.

In the case of a cannula-needle 2 with GA24 caliber, the diameter d of the peripheral holes 16 will be equal to 0.08 mm, and then the radius r will be equal to 0.04 mm. The area A of each hole 16 will therefore be equal to 0.005024 mm².

The flow rate Qi supplied by each peripheral hole 16 will be given by the formula

Qᵢ=0.61*0.005024*(2*9810*1000)=13.575 mm³/s=0.81 cm³/min

The flow rate Q_{f} delivered by the distal hole 13 will instead be equal to 29 ml/min and therefore the peripheral holes 16 along the cannula-needle 2 have to allow a flow rate equal to

Q_{iT}=(50−29) ml/min=21 ml/min

Therefore, the number of peripheral holes 16 will be

nᵢ=int(21/0.81)=25.

The actual number nₑ of holes 16 may be nₑ=34 with 17 peripheral holes for each passage, to compensate any possible errors and oversize the system.

A method for manufacturing the above cannula-needle 2 may include a step of forming the passages 15, 15′ on the peripheral wall 10 by generating and orienting a laser beam on the peripheral wall 10 itself.

In particular, when the passages 15, 15′ are defined by the peripheral holes 16 or slots 17 the step of generating and orienting the laser beam may provide a first step of adjusting the position of the tubular body 9 with respect to the generator of the laser beam by orienting a laser beam having a power less than its maximum power and with insufficient value to pierce the tubular body 9.

Subsequently the power of the laser beam will be increased to form the hole 16.

This alignment step may be carried out prior to the formation of each hole 16 or just upstream of the formation of the first hole of each passage 15, 15′.

Moreover, it may be also provided a step of adjusting the distance of the tubular body 9 from the focal point of the beam for varying the diameter of the peripheral holes 16 or the transverse dimension of the slots 17, i.e. the minimum dimension d of the passages 15, 15′.

In turn, the tubular body 9 of the cannula-needle 2 may be formed according to any of the techniques traditionally used and therefore not disclosed in more detail.

The laser generator may be selected from those commercially available, without particular limitations.

In way of an example a laser beam may be used at a wavelength of 400 nm with a repetition rate of 80 MHz, focused on the wall 10 of the cannula 2 through a lens, both mounted on two 3D translation stages for the control of position with respect to the focal point of the lens, which allows to determine the diameter d of the hole 16.

Using the translation stage, the laser will be focused on the wall 10 of the cannula 2 with a power of 1 mW, insufficient to make the holes 16, but suitable to allow the alignment of the cannula-needle 2.

Subsequently, the laser power will be increased up to a value of 160 mW for about one second, creating the hole 16.

The minimum diameter d of the hole 16 may be determined by the spot size of the selected lens and/or alternatively using a telescopic system for the laser beam to reduce its life.

A femtosecondpulsed a Ti: Sapphire laser produced by Coherent.in (Chameleon Ultra II) was used as the laser source so as to generate a laser beam with power of 4 W for a repetition rate of 80 MHz and a wavelength of 800 nm.

The beam was subsequently brought to a second harmonic generator based on crystals of barium borate to obtain a laser beam at a wavelength of 400 nm with a peak power of about 160 mW.

The second harmonic generator, in addition to generating a laser beam to the desired wavelength, will also act as a controller for the beam power.

Several lens have been used, such as lenses produced by Nikon, with numerical aperture between 0.12 and 0.60.

In view of the foregoing it appears evident that the invention achieves the intended objects and in particular to make available a cannula-needle for intravenous catheters that allows to supply maximum flow rate of greater value and more uniformly than the cannula-needle with equal size.

The cannula-needle, the infusion device and the method for making the cannula-needle according to the invention are susceptible of numerous modifications and variations, all falling within the inventive concept expressed in the appended claims. All the details may be replaced with other technically equivalent elements, and the materials may be different depending on requirements, without departing from the scope of protection of the present invention.

Even if the cannula-needle, the infusion device and method have been disclosed with particular reference to the accompanying figures, reference numbers used in the description and in the claims are used to improve the intelligence of the invention and do not constitute any limitation the claimed scope.

What is claimed is:

1. A cannula-needle configured to be associated with a catheter for intravenous injection, the catheter having a needle and a connection element to a syringe or supplying device of a fluid to be supplied, the cannula-needle has having a tubular body configured to house thereinside the needle of the catheter, the tubular body having a peripheral wall extending along a longitudinal direction (L) with a predetermined wall length (l) from an open proximal end adapted configured to be anchored to the connection element of the catheter to a distal end tapered and provided with a distal hole for the partial exit of the needle,
wherein the peripheral wall comprises at least one pair of elongated passages substantially longitudinal and substantially diametrically opposite to each other, each having a minimum dimension (d) between ⅓ and 1/7 of a maximum diameter (ID) of the distal hole and each having an overall longitudinal extension (e) greater than 50% of the predetermined wall length (l) of the peripheral wall, each of the at least one pair of elongated passages configured for obtaining a substantially uniform distribution of a supplied flow along the longitudinal extension of the tubular body.

2. The cannula-needle of claim 1, wherein the overall longitudinal extension (e) of each of the elongated passages is at least equal to 60% of the predetermined wall length (l) of the peripheral wall.

3. The cannula-needle of claim 1, wherein the minimum dimension (d) is about ⅕ of the maximum diameter (ID) of the distal hole.

4. The cannula-needle of claim 1, wherein each of the elongated passages comprises a plurality of substantially circular peripheral holes longitudinally aligned and reciprocally offset, each having a diameter defining the minimum dimension (d).

5. The cannula-needle of claim 4, wherein each of the elongated passages comprises between 15 and 25 of the substantially circular peripheral holes.

6. The cannula-needle of claim 4, wherein the minimum number of the peripheral holes is calculated with the following formula
$${n}_{i} = int ( \frac{{Q}_{t} - {Q}_{f}}{{k}_{ρ} * A * {k}_{h}} )$$
wherein Qₜ is a desired total nominal flow rate, Q_{f} is a nominal flow rate of the fluid supplied by the distal hole, A is an area of each of the peripheral holes, k_{ρ} is a parameter depending on the density of the fluid to be supplied and kₕ is a parameter calculated as a function of a falling height of the supplied fluid.

7. The cannula-needle of claim 1, wherein each of the elongated passages comprises a substantially longitudinal continuous slot having the longitudinal extension (e) and a width defining the minimum dimension.

8. The cannula-needle of claim 1, wherein each of the elongated passages comprises at least one pair of substantially longitudinal continuous slots longitudinally aligned with each other, the slots of each pair being reciprocally offset with each other and having a width defining the minimum dimension.

9. The cannula-needle of claim 1, wherein the distal hole has a hole diameter between 0.4 mm and 0.6 mm, the distal hole configured to supply a partial flow rate (Q_{f}) between 25 ml/min and 45 ml/min and a maximum total flow rate (Qₜ) between 50 ml/min and 100 ml/min, the tubular body having the predetermined wall length between 15 mm and 30 mm, the elongated passages having a minimum distance from the proximal end between 5 mm and 7.5 mm.

10. The cannula-needle of claim 1, wherein the distal hole has a diameter between mm and 0.4 mm and 0.6 mm, the distal hole configured to supply a partial flow rate (Q_{f}) between 25 ml/min and 45 ml/min and a maximum total flow rate (Qₜ) between 50 ml/min and 100 ml/min, the tubular body having a wall length between 15 mm and 30 mm, the elongated passages having a minimum distance from the distal end between 2 mm and 3 mm.

11. A method for manufacturing a cannula-needle for an intravenous catheter, the cannula-needle comprising a tubular body having a peripheral wall and a distal hole configured for supplying a fluid flow the method comprising:
forming a plurality of passages on the peripheral wall for the fluid flow, the forming carried out by generating and orienting a laser beam on the peripheral wall.

12. The method of claim 11, wherein generating and orienting the laser beam comprises:
adjusting the position of the tubular body with respect to a generator of the beam by orienting a laser beam having power less than its maximum power and with value not sufficient to pierce the tubular body; and
increasing the power of the laser beam to form the peripheral hole.

13. The method of claim 12, by further comprising:
adjusting the distance of the tubular body from the focus point of the beam to vary the diameter (d) of the peripheral hole.

14. A device, such as a catheter or the like, for intravenous injection of medical fluids, comprising:
a cannula-needle having a tubular body with an open proximal end and a distal end, the distal end tapered and provided with a distal hole and configured to be inserted into a vein of a patient;
an intravenous needle configured to be removably inserted into the tubular body through the proximal end, a tip of the intravenous needle capable of extending from the distal hole and entering the vein for supplying the medical fluid; and
a connection element configured for connecting the intravenous needle to a syringe or other infusion device.

15. The device of claim 14, wherein the connection element comprises a first portion associated with the intravenous needle and configured to connect to the syringe or infusion device, and a second portion configured to removably connect to the first portion and to be connected to the proximal end of the tubular body of the cannula-needle, the second portion comprising a hollow cylindrical body configured for allowing the passage of the intravenous needle.
